(19) United States
(12) Patent Application Publication
(10) Pub. No.: US 2012/0053092 A1
(43) Pub. Date: Mar. 1, 2012

(54) Shale Hydration Inhibition Agents for Utilization in Water-based Drilling Fluids

(75) Inventors: D. Gerrard Marangoni, Antigonish (CA); James Nyangulu, Antigonish (CA); Sean Gillis, Antigonish (CA); Amanda MacInnis, Antigonish (CA); Aleisha McLachlan, Antigonish (CA); Josette Landry, Antigonish (CA)

(73) Assignee: St. Francis Xavier University, Antigonish (CA)

(21) Appl. No.: 13/123,349

(22) PCT Filed: Oct. 9, 2009
(86) PCT No.: PCT/CA2009/001432

§ 371 Date: Nov. 4, 2011

Related U.S. Application Data
(60) Provisional application No. 61/195,649, filed on Oct. 9, 2008.

(51) Int. Cl. C09K 8/04; C07C 215/18; C07C 211/64; C07C 211/62

(52) U.S. Cl. 507/131; 507/129; 564/281; 564/292; 564/295

(57) ABSTRACT

Shale hydration inhibition agents of the general formula: wherein R1, R2, R3, R4, R5 and R6 are as defined herein, are described. In addition, the present application is directed to water-based drilling fluids containing these shale hydration inhibition agents and methods of using such agents to inhibit the hydration or swelling of shale during drilling.

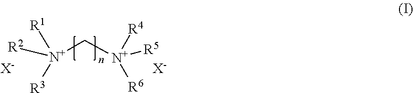

### FIELD OF THE INVENTION

The present invention relates to drilling fluids; specifically to water based drilling fluids containing one or more agents to reduce or eliminate shale swelling in shale containing formations which are prone to hydration and swelling (hydratable shales). The invention also relates to agents which inhibit the hydration and swelling of shale.

### BACKGROUND OF THE INVENTION

When subterranean oil wells are drilled, drilling fluids are generally used to cool and lubricate the rotary drill bit, to carry cuttings to the surface and to stabilize shale formations through which the well bore is being drilled. A problem associated with oil well drilling is swelling of clays associated with shale formations, which can significantly impede the performance of the drill, leading to increased drilling times and increased costs. This swelling can occur when clay minerals in the formation absorb water, which is then positioned between adjacent layers within the crystalline structure of the clay, causing an increase in the inter-layer spacing (commonly referred to as “c-spacing”).

Attempts to address this problem include adding shale inhibitors, also known as shale hydration inhibitors, to the drilling fluid. A wide variety of shale inhibitors have been used in drilling fluids. For example, amine or ammonium compounds have been reported as shale inhibitors or drilling fluid additives in U.S. Pat. Nos. 6,247,543; 6,291,406; 6,484,821; 6,609,578; 6,831,043; 6,857,485; and 7,012,043; and US Published Applications No. 2002/0155956; 2005/0049149; 2005/0049150; 2005/0096232; 2006/0073982; 2006/0073983; 2006/0073984; 2006/0073985; 2006/0116296; 2006/0137878; 2006/0194700; 2007/0082823; and 2007/0129258. However many of the known shale inhibitors have a low solubility in water and therefore are not desirable for use in water-based drilling fluids.

### SUMMARY OF THE INVENTION

One aspect of the present invention provides shale hydration inhibition agents which are compounds of formula I:

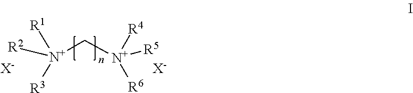

wherein R¹, R², R³, R⁴, R⁵ and R⁶ are each independently selected from hydrogen, (C₁₋₆)alkyl, (C₂₋₆)alkenyl, (C₂₋₆)alkynyl, (C₃₋₇)cycloalkyl, hydroxy(C₁₋₆)alkyl, (C₁₋₆)alkoxy(C₁₋₆)alkyl, aryl(C₁₋₆)alkyl, and (C₁₋₆)alkylamido(C₁₋₆)alkyl;
n is an integer from 1 to 6; and
X⁻ is a counterion;
provided that when n is 6, at least one of R¹, R², R³, R⁴, R⁵ and R⁶ is not hydrogen.

Another aspect of the present invention provides the use of a compound of formula I as a shale hydration inhibition agent.

Another aspect of the present invention provides a water-based drilling fluid for use in drilling through a formation containing shale, wherein the drilling fluid comprises a compound of formula I.

Another aspect of the present invention provides a method of inhibiting the hydration and swelling of shale when drilling through a formation containing shale, the method comprising using a water based drilling fluid comprising a compound of formula I.

### DEFINITIONS

The term “substituent”, as used herein and unless specified otherwise, is intended to mean an atom, radical or group which may be bonded to a carbon atom, a heteroatom or any other atom which may form part of a molecule or fragment thereof, which would otherwise be bonded to at least one hydrogen atom. Substituents contemplated in the context of a specific molecule or fragment thereof are those which give rise to chemically stable compounds, such as are recognized by those skilled in the art.

The terms “alkyl” or “(C₁₋ₙ)alkyl” as used herein, wherein n is an integer, either alone or in combination with another radical, are intended to mean an acyclic, straight or branched chain alkyl radical containing from 1 to n carbon atoms. “Alkyl” includes, but is not limited to, methyl, ethyl, propyl (n-propyl), butyl (n-butyl), 1-methylethyl (iso-propyl), 1-methylpropyl (sec-butyl), 2-methylpropyl (iso-butyl), 1,1-dimethylethyl (tert-butyl), pentyl and hexyl. The abbreviation Me denotes a methyl group; Et denotes an ethyl group, Pr denotes a propyl group, iPr denotes a 1-methylethyl group, Bu denotes a butyl group and tBu denotes a 1,1-dimethylethyl group.

The terms “alkenyl” or “(C₂₋ₙ)alkenyl”, as used herein, wherein n is an integer, either alone or in combination with another radical, are intended to mean an unsaturated, acyclic straight or branched chain radical containing two to n carbon atoms, at least two of which are bonded to each other by a double bond. Examples of such radicals include, but are not limited to, ethenyl (vinyl), 1-propenyl, 2-propenyl, and 1-butenyl. Unless specified otherwise, the term “(C₂₋ₙ)alkenyl” is understood to encompass individual stereoisomers where possible, including but not limited to (E) and (Z) isomers, and mixtures thereof. When a (C₂₋ₙ)alkenyl group is substituted, it is understood to be substituted on any carbon atom thereof which would otherwise bear a hydrogen atom, unless specified otherwise, such that the substitution would give rise to a chemically stable compound.

The terms “alkynyl” or “(C₂₋ₙ)alkynyl”, as used herein, wherein n is an integer, either alone or in combination with another radical, are intended to mean an unsaturated, acyclic straight or branched chain radical containing two to n carbon atoms, at least two of which are bonded to each other by a triple bond. Examples of such radicals include, but are not limited to, ethynyl, 1-propynyl, 2-propynyl, and 1-butynyl. When a (C₂₋ₙ)alkynyl group is substituted, it is understood to be substituted on any carbon atom thereof which would otherwise bear a hydrogen atom, unless specified otherwise, such that the substitution would give rise to a chemically stable compound.

The term “cycloalkyl” or “(C₃₋ₘ)cycloalkyl” as used herein, wherein m is an integer, either alone or in combination with another radical, are intended to mean a cycloalkyl substituent containing from 3 to m carbon atoms and includes, but is not limited to, cyclopropyl, cyclobutyl, cyclopentyl, cyclohexyl and cycloheptyl.

The terms “alkoxy” or “(C₁₋ₙ)alkoxy” as used herein, wherein n is an integer, either alone or in combination with another radical, are intended to mean an oxygen atom further bonded to an alkyl group containing 1 to n carbon atoms as defined above. “Alkoxy” includes, but is not limited to, methoxy (—OCH₃), ethoxy (—OCH₂CH₃), propoxy (—OCH₂CH₂CH₃), butoxy (—OCH₂CH₂CH₂CH₃), 1-methylethoxy (—OCH(CH₃)₂), and 1,1-dimethylethoxy (—OC(CH₃)₃).

The term “aryl” as used herein, either alone or in combination with another radical, is intended to mean a carbocyclic aromatic monocyclic group containing 6 carbon atoms which may be further fused to a second 5- or 6-membered carbocyclic group which may be aromatic, saturated or unsaturated. “Aryl” includes, but is not limited to, phenyl, indanyl, indenyl, 1-naphthyl, 2-naphthyl, tetrahydronaphthyl and dihydronaphthyl. An aryl group can optionally be substituted with from 1 to 5 substituents each independently chosen from substituents known in the art, including but not limited to (C₁₋₆)alkyl, (C₂₋₆)alkenyl, (C₂₋₆)alkynyl, halogen, —NO₂, and —OH.

The terms “arylalkyl” or “aryl(C₁₋ₙ)alkyl” as used herein, wherein n is an integer, either alone or in combination with another radical, are intended to mean an alkyl radical having 1 to n carbon atoms as defined above which is itself substituted with an aryl radical as defined above. Examples of arylalkyl include, but are not limited to, phenylmethyl (benzyl), 1-phenylethyl, 2-phenylethyl and phenylpropyl. When an arylalkyl group is substituted, it is understood that substituents may be attached to either the aryl or the alkyl portion thereof or both, unless specified otherwise, such that the substitution would give rise to a chemically stable compound, such as are recognized by those skilled in the art.

The terms “alkylamidoalkyl” or “(C₁₋ₙ)alkylamido(C₁₋ₙ)alkyl” as used herein, wherein n is an integer, either alone or in combination with another radical, are intended to mean radicals of the formula (C₁₋ₙ)alkyl-C(═O)—NH—(C₁₋ₙ)alkyl- or (C₁₋ₙ)alkyl-NHC(═O)—(C₁₋ₙ)alkyl-.

### DETAILED DESCRIPTION OF THE INVENTION

In at least one embodiment, the shale hydration inhibition agent according to the present invention is a bis-surfactant diamine compound (commonly referred to as a “Gemini surfactant”). Such surfactants can be prepared according to a number of methods as disclosed in standard organic chemistry textbooks and publications such as the Kirk-Othmer Encyclopedia of Chemical Technology. For ease of reference, these molecules are described and designated below by the working product name X-Gem Inhibitors.

In at least one embodiment, the present invention provides shale hydration inhibition agents of the formula I:

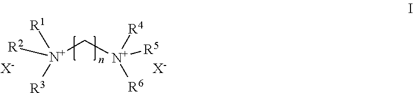

wherein R¹, R², R³, R⁴, R⁵ and R⁶ are each independently selected from hydrogen, (C₁₋₆)alkyl, (C₂₋₆)alkenyl, (C₂₋₆)alkynyl, (C₃₋₇)cycloalkyl, hydroxy(C₁₋₆)alkyl, (C₁₋₆)alkoxy(C₁₋₆)alkyl, aryl(C₁₋₆)alkyl, and (C₁₋₆)alkylamido(C₁₋₆)alkyl;
n is an integer from 1 to 6; and
X⁻ is a counterion;
provided that when n is 6, at least one of R¹, R², R³, R⁴, R⁵ and R⁶ is not hydrogen.

In at least one embodiment, the group —N⁺(R¹)(R²)(R³) is the same as the group —N⁺(R⁴)(R⁵)(R⁶); provided that when n is 6, at least one of R¹, R², R³, R⁴, R⁵ and R⁶ is not hydrogen.

In at least one embodiment, the group —N⁺(R¹)(R²)(R³) is different from the group —N⁺(R⁴)(R⁵)(R⁶); provided that when n is 6, at least one of R¹, R², R³, R⁴, R⁵ and R⁶ is not hydrogen.

In at least one embodiment, R¹, R², R³, R⁴, R⁵ and R⁶ are each independently selected from hydrogen, (C₁₋₄)alkyl, (C₂₋₄)alkenyl, (C₂₋₄)alkynyl, (C₃₋₆)cycloalkyl, hydroxy(C₁₋₄)alkyl, (C₁₋₄)alkoxy(C₁₋₄)alkyl, aryl(C₁₋₄)alkyl, and (C₁₋₄)alkylamido(C₁₋₄)alkyl; provided that when n is 6, at least one of R¹, R², R³, R⁴, R⁵ and R⁶ is not hydrogen.

In at least one embodiment, R¹, R², R³, R⁴, R⁵ and R⁶ are each independently selected from hydrogen, (C₁₋₂)alkyl, (C₂)alkenyl, (C₂)alkynyl, (C₃₋₄) cycloalkyl, hydroxy(C₁₋₂)alkyl, (C₁₋₂)alkoxy(C₁₋₂)alkyl, aryl(C₁₋₂)alkyl, and (C₁₋₂)alkylamido(C₁₋₂)alkyl; provided that when n is 6, at least one of R¹, R², R³, R⁴, R⁵ and R⁶ is not hydrogen.

In at least one embodiment, R¹, R², R³, R⁴, R⁵ and R⁶ are each independently selected from hydrogen, methyl, ethyl, cyclohexyl, benzyl, hydroxyethyl and hydroxypropyl; provided that when n is 6, at least one of R¹, R², R³, R⁴, R⁵ and R⁶ is not hydrogen.

In at least one embodiment, n is an integer from 1 to 4.

In at least one embodiment, n is an integer selected from 1, 2, 4 and 5.

In at least one embodiment, X⁻ is a counterion selected from bromide, chloride, iodide, hydroxide, a carboxylate including but not limited to acetate, formate, and propionate, a sulfonate including but not limited to methanesulfonate (mesylate), ethanesulfonate, trifluoromethanesulfonate (triflate), benzenesulfonate (besylate), p-toluenesulfonate (tosylate), p-nitrobenzenesulfonate (nosylate), and p-bromobenzenesulfonate (brosylate), and other anionic counterions known in the art.

It will be apparent to the person of skill in the art that when at least one of R¹, R², R³, R⁴, R⁵ and R⁶ is hydrogen, the shale hydration inhibition agents of formula I will exist in pH-dependent equilibrium with unprotonated forms. For example, when R¹ is H, compounds of formula Ia and IIa will exist in pH-dependent equilibrium as illustrated by the equation below.

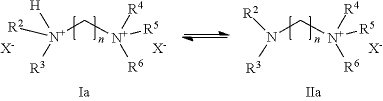

Furthermore, when R¹ and R⁴ are both H, compounds of formula Ib, IIb, IIIb and IVb will exist in pH-dependent equilibrium as illustrated by the equation below.

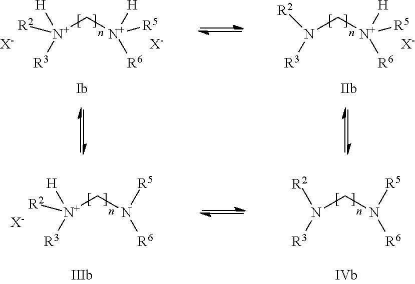

The person of skill in the art will recognize that analogous pH-dependent equilibria are possible whenever any one or more of R¹, R², R³, R⁴, R⁵ and R⁶ is hydrogen. It is contemplated that the shale hydration inhibition agents of formula I include compounds of formulas Ia, IIa, Ib, IIb, IIIb and IVb and analogous species formed in pH-dependent equilibria whenever any one or more of R¹, R², R³, R⁴, R⁵ and R⁶ is hydrogen.

One aspect of the present invention provides a water-based drilling fluid for use in drilling through a formation containing shale, wherein the drilling fluid comprises a shale hydration inhibition agent of formula I. In at least one embodiment, the shale hydration inhibition agent is present in sufficient concentration to reduce swelling of the shale while drilling is carried out.

In at least one embodiment, the drilling fluid further comprises at least one weight material and an aqueous continuous phase. A weight material is an inert, high-density particulate material used to increase the density of the drilling fluid. Suitable weight materials are known in the art and include, but are not limited to such examples as calcium carbonate, magnesium carbonate, iron oxide, barite, hematite, ilmenite, water-soluble organic and inorganic salts, and mixtures thereof.

In at least one embodiment, the drilling fluid comprises one or more additional components which may be added to an aqueous based drilling fluid, including but not limited to fluid loss control agents, bridging agents, lubricants, anti-bit balling agents, corrosion inhibition agents, surfactants and suspending agents. Such components can be added in the concentrations needed to adjust the rheological and functional properties of the drilling fluid appropriate to the drilling conditions, as would be apparent to the skilled person. Suitable examples of each of these additional components are well known to the person of skill in the art.

Fluid loss control agents are added to drilling fluids to help prevent or reduce fluid loss during the drilling process. Suitable examples of fluid loss control agents include but are not limited to synthetic organic polymers including but not limited to polyacrylate; biopolymers including but not limited to starches, modified starches and modified celluloses; modified lignite; lignosulfonate; silica; mica; calcite; and mixtures thereof.

Bridging agents are materials added to a drilling fluid to bridge across pores and fractures of exposed rock, to seal formations, and to aid in forming a filter cake. Advantageously, bridging agents are removable from the wellbore after drilling is complete, to facilitate recovery when the well enters production. Suitable examples of bridging agents include but are not limited to magnesium oxide, manganese oxide, calcium oxide, lanthanum oxide, cupric oxide, zinc oxide, magnesium carbonate, calcium carbonate, zinc carbonate, calcium hydroxide, manganese hydroxide, suspended salts, oil-soluble resins, mica, nutshells, fibers and mixtures thereof.

Lubricants are used to lower friction, including but not limited to torque and drag in the wellbore, and to lubricate unsealed bit bearings. Suitable examples of lubricants include but are not limited to plastic beads, glass beads, nut hulls, graphite, oils, synthetic fluids, glycols, modified vegetable oils, fatty-acid soaps, surfactants and mixtures thereof.

Anti-bit balling agents are used to prevent compaction and adherence of drill cuttings to the cutting surfaces of the drill bit, causing fouling and a reduction of drill performance. Suitable examples of anti-bit balling agents include but are not limited to glycols, surfactants and mixtures thereof.

Corrosion inhibition agents are used to protect the metal components of the drill from corrosion caused by contact with materials such as water, carbon dioxide, biological deposits, hydrogen sulfide and acids. Suitable examples of corrosion inhibition agents include but are not limited to amines, zinc compounds, chromate compounds, cyanogen-based inorganic compounds, sodium nitrite based compounds and mixtures thereof.

Surfactants are surface active agents that can function as emulsifiers, dispersants, oil-wetters, water-wetters, foamers and defoamers. Suitable examples of surfactants include but are not limited to anionic surfactants, cationic surfactants, zwitterionic surfactants, nonionic surfactants, and suitable mixtures of any of the above known to one skilled in the art.

Suspending agents alter the rheological and viscosity properties of the drilling fluid, thereby allowing small solid particles to remain suspended in the fluid. Suitable examples of suspending agents include but are not limited to clays, biopolymers, gums, silicates, fatty acids, synthetic polymers and mixtures thereof.

### Synthetic Methodology

The shale inhibition agents of formula (I)

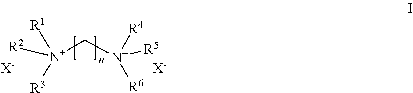

wherein the group —N⁺(R¹)(R²)(R³) is the same as the group —N⁺(R⁴)(R⁵)(R⁶) and wherein R¹, R², R³, R⁴, R⁵, R⁶ and X are as defined herein, are conveniently prepared by the procedure outlined in Scheme 1.

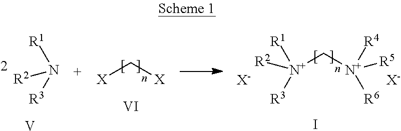

A mixture of reactants V and VI, wherein R¹, R² and R³ are as defined herein, R⁴ is R¹, R⁵ is R², R⁶ is R³, and X is a leaving group which gives rise to the counterion X⁻, are allowed to react in a molar ratio of at least 2:1, in an appropriate solvent under reflux, to provide compound I wherein the group —N⁺(R¹)(R²)(R³) is the same as the group —N⁺(R⁴)(R⁵)(R⁶) and wherein R¹, R², R³, R⁴, R⁵, R⁶ and X are as defined herein.

Furthermore, the shale inhibition agents of formula (I)

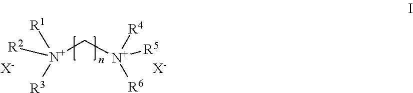

wherein the group —N⁺(R¹)(R²)(R³) is different from the group —N⁺(R⁴)(R⁵)(R⁶) and wherein R¹, R², R³, R⁴, R⁵, R⁶ and X are as defined herein, are conveniently prepared by the procedure outlined in Scheme 2.

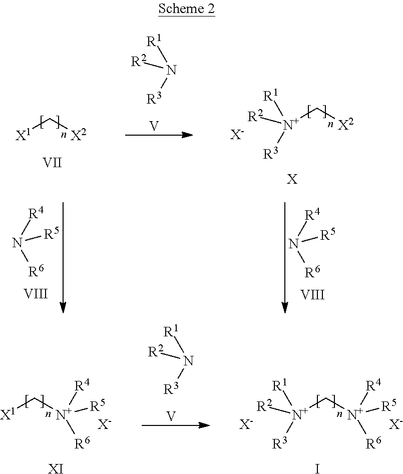

X¹ and X² are leaving groups or groups which may be transformed to leaving groups, as will be recognized by the person of skill in the art. X¹ and X² are chosen so that reagent VII can be reacted sequentially, in either order, with amine reagents V and VIII, to give intermediates X or XI, each of which can then be transformed to compounds of formula I wherein R¹, R², R³, R⁴, R⁵, R⁶ and X are as defined herein, using reactions well known in the art.

It will be apparent to the skilled person that, in addition to the procedures described herein, the surfactants of the present invention can be prepared according to a number of methods as disclosed in standard organic chemistry textbooks and publications such as the Kirk-Othmer Encyclopedia of Chemical Technology.

### EXAMPLES

Other features of the present invention will become apparent from the following non-limiting examples which illustrate, by way of example, the principles of the invention. It will be apparent to a skilled person that the procedures exemplified below may be used, with appropriate modifications, to prepare other shale hydration inhibition agents of the invention as described herein.

### Example 1

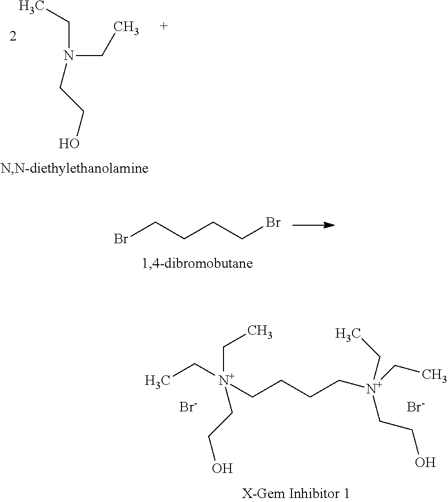

X-Gem Inhibitor 1 is prepared by combining N,N-diethylethanolamine (105 mL) with 1,4-dibromobutane (60 mL) in a reaction vessel. Dichloromethane is added to a total volume of about 350 mL. The mixture is heated at reflux for a period of several days. Upon completion of the reaction, the dichloromethane is removed and the reaction product named X-Gem Inhibitor 1 is recovered in good yield as a reddish-orange liquid. The product of the reaction is characterized by ¹H and ¹³C NMR spectroscopy and infrared spectroscopy.

### Example 2

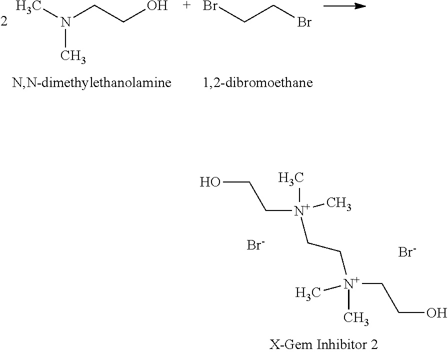

X-Gem Inhibitor 2 is prepared by combining N,N-dimethylethanolamine (21.1 mL) with 1,2-dibromoethane (8.6 mL) in a reaction vessel. Dichloromethane is added to a total volume of about 320 mL. The mixture is heated at reflux for a period of several days. Upon completion of the reaction, the dichloromethane is removed and the reaction product named X-Gem Inhibitor 2 is recovered in good yield as a dark liquid. The product of the reaction is characterized by ¹H and ¹³C NMR spectroscopy and infrared spectroscopy.

### Example 3

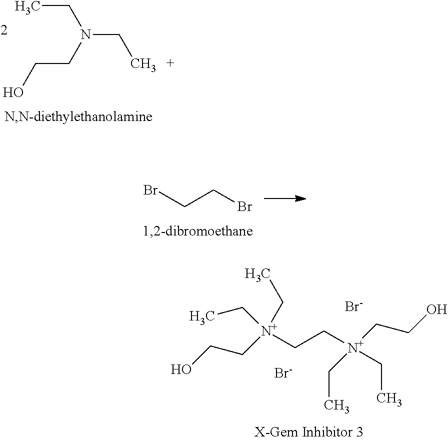

X-Gem Inhibitor 3 is prepared by combining N,N-diethylethanolamine (63.3 mL) with 1,2-dibromoethane (25.8 mL) in a reaction vessel. Dichloromethane is added to a total volume of about 270 mL. The mixture is heated at reflux for a period of several days. Upon completion of the reaction, the dichloromethane is removed and the reaction product named X-Gem Inhibitor 3 is recovered in good yield as a slightly orange liquid. The product of the reaction is characterized by ¹H and ¹³C NMR spectroscopy and infrared spectroscopy.

### Example 4

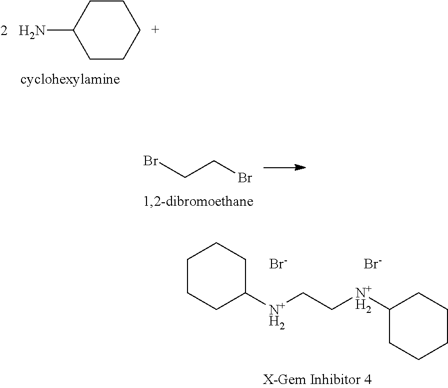

X-Gem Inhibitor 4 is prepared by combining cyclohexylamine (24 mL) with 1,2-dibromoethane (11.8 mL) in a reaction vessel. Dichloromethane is added to a total volume of about 300 mL. The mixture is heated at reflux for a period of several days. Upon completion of the reaction, the dichloromethane is removed and the reaction product named X-Gem Inhibitor 4 is recovered in good yield as a slightly yellowish solid. The product is characterized by ¹H and ¹³C NMR spectroscopy and infrared spectroscopy.

### Example 5

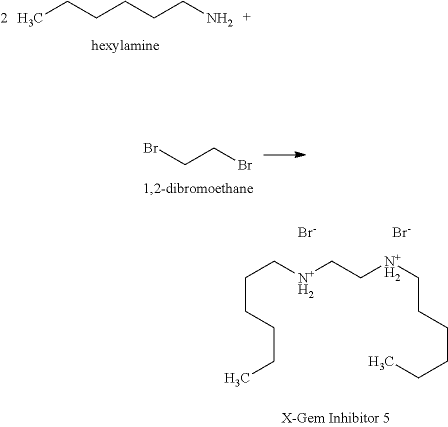

X-Gem Inhibitor 5 is prepared by combining hexylamine (27.7 mL) with 1,2-dibromoethane (11.8 mL) in a reaction vessel. Dichloromethane is added to a total volume of about 310 mL. The mixture is heated at reflux for a period of several days. Upon completion of the reaction, the dichloromethane is removed and the reaction product named X-Gem Inhibitor 5 is recovered in good yield as a slightly yellowish liquid. The product is characterized by ¹H and ¹³C NMR spectroscopy and infrared spectroscopy.

### Example 6

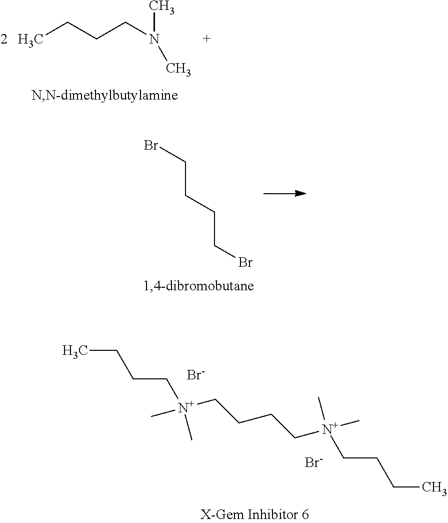

X-Gem Inhibitor 6 is prepared by combining N,N-dimethylbutylamine (21.4 mL) with 1,4-dibromobutane (8.2 mL) in a reaction vessel. Dichloromethane is added to a total volume of about 300 mL. The mixture is heated at reflux for a period of several days. Upon completion of the reaction, the dichloromethane is removed and the reaction product named X-Gem Inhibitor 6 is recovered in good yield as a white crystalline solid. The product is characterized by ¹1H and ¹³C NMR spectroscopy, infrared spectroscopy and mass spectrometry.

### Example 7

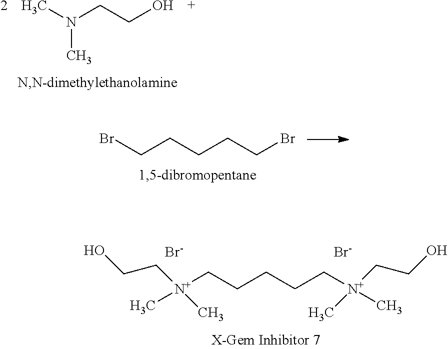

X-Gem Inhibitor 7 is prepared by combining N,N-dimethylethanolamine (16.0 mL) with 1,5-dibromopentane (9.0 mL) in a reaction vessel. Acetonitrile is added to a total volume of about 300 mL. The mixture is heated at reflux for a period of several days. Upon completion of the reaction, the acetonitrile is removed and the reaction product named X-Gem Inhibitor 7 is recovered in good yield as a slightly yellowish, crystalline solid. The product is characterized by ¹H and ¹³C NMR spectroscopy and infrared spectroscopy.

### Analysis

Test results for lab formulations containing 5 examples of the present invention described above are discussed below. Shale dispersion (hot roll dispersion) tests are performed as described below.

A wash solution of 42.75 kg/m³ (15 lb/bbl) KCl brine is prepared by adding 85.5 g of KCl to 2 L of triply deionized water and mixing for 15 minutes. Solutions are prepared by adding specific concentrations of the inhibitor (units of L/m³ for liquids; kg/m³ for solids) to 350 mL of water and mixing on a Hamilton Beach mixer (low setting—50% variac) for 15 minutes. These solutions are allowed to stand for 1 hour to hydrate. To those solutions, 10 g samples of Pierre 2 shale (retained on 16 mesh screen after being sieved through a 10 mesh screen, weighed to +/−0.01 g on a calibrated balance) are added to the samples in hot roll cells. The samples are hot-rolled for 16 hours at 150° F. After hot rolling is completed, the solutions are passed through 10, 16, and 40 mesh screens while rinsing gently with the KCl brine prepared above. After washing, all three screens are immersed together into fresh cold water for approximately one minute. This is repeated an additional two times. Shale samples are dried to a consistent weight in an oven at 105° C. (220° F.). The samples are re-weighed on the screen(s) on the calibrated digital balance and the measured weights are used to obtain the percent shale recovery (PSR) as follows

PSR=(a+b+c)/d*100

where: a is the mass of dry shale on the 10 mesh screen, b is the mass of dry shale on the 16 mesh screen, c is the mass of dry shale on the 40 mesh screen, and d is the initial mass of shale added to the solutions.

Testing of the inhibitors is done with inhibitor, water and shale as well as in a drilling fluid system. The inhibitors used were X-Gem Inhibitor 1, X-Gem Inhibitor 2, X-Gem Inhibitor 3, X-Gem Inhibitor 4, X-Gem Inhibitor 5, HighPerm™ (Newpark Drilling Fluids, Calgary, Alberta) and a 4% glycol/KCl (70 kg/m³) solution, and a control with no inhibitor is also tested. In addition, shale dispersion tests are carried out with the drilling fluid system EZ Clean™ (Newpark Drilling Fluids, Calgary, Alberta) as a control/reference, and the known shale inhibitor HighPerm™ in the EZ Clean™ system is replaced with the X-Gem inhibitors, to identify any potential adverse effects that could render the fluid ineffective as an inhibitor to clay swelling.

A series of samples (350 mL) are made up with 10 g of shale cuttings and either water or EZ Clean™ containing various concentrations of shale inhibitor. The samples are hot rolled for 16 hours at 150° F. The results are presented in Table 1. BHR refers to “before hot rolling” while AHR refers to “after hot rolling”. PSR refers to percent shale recovery obtained using a 16 mesh screen for shale recovery.

TABLE 1

| | | | Shale | |
|---|---|---|---|---|
| | | Shale | AHR | |
| Compound | Concentration | BHR (g) | (g) | PSR |
| X-Gem Inhibitor 1 in water | 80 L/m³ | 10.0 | 9.8 | 98 |
| X-Gem Inhibitor 1 in water | 40 L/m³ | 10.0 | 9.6 | 96 |
| X-Gem Inhibitor 2 in water | 40 L/m³ | 10.0 | 9.7 | 97 |
| X-Gem Inhibitor 3 in water | 40 L/m³ | 10.0 | 9.4 | 94 |
| X-Gem Inhibitor 4 in water | 10 kg/m³ | 10.0 | 9.6 | 96 |
| X-Gem Inhibitor 5 in water | 40 L/m³ | 10.0 | 9.4 | 94 |
| X-Gem Inhibitor 6 in water | 10 kg/m³ | 10.0 | 9.5 | 95 |
| X-Gem Inhibitor 7 in water | 10 L/m³ | 10.0 | 9.3 | 93 |
| HighPerm ™ in water | 80 L/m³ | 10.0 | 9.6 | 96 |
| HighPerm ™ in water | 40 L/m³ | 10.0 | 9.0 | 90 |
| Glycol/KCl (70 kg/m³) in | 40 L/m³ | 10.0 | 9.7 | 97 |
| water | | | | |
| EZ Clean ™ with | 5 L/m³ | 10.0 | 9.3 | 93 |
| HighPerm ™ | | | | |
| EZ Clean ™ with X-Gem | 5 L/m³ | 10.0 | 9.6 | 96 |
| Inhibitor 1 | | | | |
| EZ Clean ™ with X-Gem | 5 L/m³ | 10.0 | 9.8 | 98 |
| Inhibitor 2 | | | | |
| EZ Clean ™ with X-Gem | 5 L/m³ | 10.0 | 9.6 | 96 |
| Inhibitor 3 | | | | |
| EZ Clean ™ with X-Gem | 10 kg/m³ | 10.0 | 9.8 | 98 |
| Inhibitor 4 | | | | |
| EZ Clean ™ with X-Gem | 5 L/m³ | 10.0 | 9.7 | 97 |
| Inhibitor 5 | | | | |
| Water | | 10.0 | 1.2 | 12 |

As can be seen from the results in Table 1, X-Gem Inhibitor 1, X-Gem Inhibitor 2, X-Gem Inhibitor 3, X-Gem Inhibitor 4 and X-Gem Inhibitor 5 are very comparable as shale hydration inhibitors to the HighPerm™ and Glycol/KCl solution. The drilling fluid system with EZ Clean™ and the X-Gem inhibitors have a very high percent shale recovery (PSR). It should be noted that the fluids that include the X-Gem inhibitors all have higher PSR than the EZ Clean™ system.

X-Gem Inhibitor 1, X-Gem Inhibitor 2, X-Gem Inhibitor 3, X-Gem Inhibitor 4 and X-Gem Inhibitor 5 are used to replace the known shale inhibitor HighPerm™ in a known drilling fluid system (EZ Clean™) to determine the effect on the rheological properties compared to those of the EZ Clean™ system containing HighPerm™ Rheological measurements are carried out on an OFI Model 900 rheometer at 25° C. and 50° C. The results can be seen in Tables 2 to 6. In the Tables below, the following terminology is used to describe the rheological behaviour of the fluids. Unless otherwise stated, all starting materials are commercially available and standard laboratory techniques are used. The tests are conducted in accordance with the procedures in API Bulletin RP 13B-1, Third Edition, 2003. The following abbreviations are used in describing the results discussed in the Tables. Dial readings at a specified shear rate are symbolized by θᵣ where r is the rpm reading. The plastic viscosity (mPa·s) is the slope of the shear stress/shear rate curve above its yield point, and is calculated by subtracting the dial reading at 300 rpm from the dial reading at 600 rpm. The yield point is the shear stress of the fluid extrapolated to 0 shear rate. The yield point is calculated in Pa as follows.

$$YP = \frac{{θ}_{300} - PV}{2}$$

The yield point (lbs/100 ft²) is found from =2×yield point (Pa)). The difference between the 10 second gel strength and the 10 minute gel strength (both in lbs/100 ft²) indicates the suspending characteristics and the thixotropic properties of a drilling fluid.

TABLE 2

| | | X-Gem Inhibitor 1 | | | |
|---|---|---|---|---|---|
| | | (Fann Dial | | EZ Clean ™ | |
| | | Readings) | | (Fann Dial Readings) | |
| | | 25° C. | 50° C. | 25° C. | 50° C. |
| Shear rate | θ₆₀₀ | 35 | 24 | 34 | 24 |
| (min⁻¹) | θ₃₀₀ | 25 | 18 | 24 | 17 |
| | θ₂₀₀ | 21 | 14 | 20 | 13 |
| | θ₁₀₀ | 15 | 9 | 14 | 9 |
| | θ₆ | 3 | 2 | 3 | 1 |
| | θ₃ | 2 | 1 | 2 | 1 |

| | | | | |
|---|---|---|---|---|
| 10 sec gel (lbs/100 ft²) | 3 | 2 | 2 | 1 |
| 10 min gel (lbs/100 ft²) | 3 | 2 | 2 | 1 |
| Plastic Viscosity (mPa · s) | 10 | 6 | 10 | 7 |
| Yield Point (lbs/100 ft²) | 7.5 | 6 | 7 | 5 |

The results show that the substitution of X-Gem Inhibitor 1 into the drilling fluid system does not have an adverse effect on rheology. The results with X-Gem Inhibitor 1 and EZ Clean™ are virtually identical.

TABLE 3

| | | X-Gem Inhibitor 2 | | | |
|---|---|---|---|---|---|
| | | (Fann Dial | | EZ Clean ™ | |
| | | Readings) | | (Fann Dial Readings) | |
| | | 25° C. | 50° C. | 25° C. | 50° C. |
| Shear rate | θ₆₀₀ | 39 | 29 | 34 | 24 |
| (min⁻¹) | θ₃₀₀ | 28 | 21 | 24 | 17 |
| | θ₂₀₀ | 24 | 16 | 20 | 13 |
| | θ₁₀₀ | 16 | 11 | 14 | 9 |
| | θ₆ | 4 | 2 | 3 | 1 |
| | θ₃ | 3 | 1 | 2 | 1 |

| | | | | |
|---|---|---|---|---|
| 10 sec gel (lbs/100 ft²) | 3 | 2 | 2 | 1 |
| 10 min gel (lbs/100 ft²) | 3 | 2 | 2 | 1 |
| Plastic Viscosity (mPa · s) | 11 | 8 | 10 | 7 |
| Yield Point (lbs/100 ft²) | 8.5 | 6.5 | 7 | 5 |

The results show that the substitution of X-Gem Inhibitor 2 into the drilling fluid system causes the rheology to increase slightly.

TABLE 4

| | | X-Gem Inhibitor 3 | | | |
|---|---|---|---|---|---|
| | | (Fann Dial | | EZ Clean ™ | |
| | | Readings) | | (Fann Dial Readings) | |
| | | 25° C. | 50° C. | 25° C. | 50° C. |
| Shear rate | θ₆₀₀ | 43 | 31 | 34 | 24 |
| (min⁻¹) | θ₃₀₀ | 32 | 23 | 24 | 17 |
| | θ₂₀₀ | 27 | 18 | 20 | 13 |
| | θ₁₀₀ | 19 | 13 | 14 | 9 |
| | θ₆ | 4 | 3 | 3 | 1 |
| | θ₃ | 3 | 2 | 2 | 1 |

| | | | | |
|---|---|---|---|---|
| 10 sec gel (lbs/100 ft²) | 3 | 2 | 2 | 1 |
| 10 min gel (lbs/100 ft²) | 3 | 2 | 2 | 1 |
| Plastic Viscosity (mPa · s) | 11 | 8 | 10 | 7 |
| Yield Point (lbs/100 ft²) | 10.5 | 7.5 | 7 | 5 |

The results show that the substitution of X-Gem Inhibitor 3 into the drilling fluid system has a minor effect on the rheology.

TABLE 5

| | | X-Gem Inhibitor 4 | | | |
|---|---|---|---|---|---|
| | | (Fann Dial | | EZ Clean ™ | |
| | | Readings) | | (Fann Dial Readings) | |
| | | 25° C. | 50° C. | 25° C. | 50° C. |
| Shear rate | θ₆₀₀ | 40 | 29 | 34 | 24 |
| (min⁻¹) | θ₃₀₀ | 29 | 21 | 24 | 17 |
| | θ₂₀₀ | 24 | 17 | 20 | 13 |
| | θ₁₀₀ | 17 | 11 | 14 | 9 |
| | θ₆ | 4 | 2 | 3 | 1 |
| | θ₃ | 3 | 1 | 2 | 1 |

| | | | | |
|---|---|---|---|---|
| 10 sec gel (lbs/100 ft²) | 3 | 1 | 2 | 1 |
| 10 min gel (lbs/100 ft²) | 3 | 1 | 2 | 1 |
| Plastic Viscosity (mPa · s) | 11 | 8 | 10 | 7 |
| Yield Point (lbs/100 ft²) | 9 | 6.5 | 7 | 5 |

The results show that the substitution of X-Gem Inhibitor 4 into the drilling fluid system causes the rheology to increase slightly and is virtually the same as the fluid with X-Gem Inhibitor 2.

TABLE 6

| | | X-Gem Inhibitor 5 | | | |
|---|---|---|---|---|---|
| | | (Fann Dial | | EZ Clean ™ | |
| | | Readings) | | (Fann Dial Readings) | |
| | | 25° C. | 50° C. | 25° C. | 50° C. |
| Shear rate | θ₆₀₀ | 43 | 32 | 34 | 24 |
| (min⁻¹) | θ₃₀₀ | 32 | 24 | 24 | 17 |
| | θ₂₀₀ | 27 | 19 | 20 | 13 |
| | θ₁₀₀ | 19 | 13 | 14 | 9 |
| | θ₆ | 5 | 3 | 3 | 1 |
| | θ₃ | 4 | 2 | 2 | 1 |

| | | | | |
|---|---|---|---|---|
| 10 sec gel (lbs/100 ft²) | 4 | 3 | 2 | 1 |
| 10 min gel (lbs/100 ft²) | 4 | 2 | 2 | 1 |
| Plastic Viscosity (mPa · s) | 11 | 8 | 10 | 7 |
| Yield Point (lbs/100 ft²) | 10.5 | 8 | 7 | 5 |

The results show that the substitution of X-Gem Inhibitor 5 into the drilling fluid system causes the rheology to increase slightly and is virtually the same as the fluid with X-Gem Inhibitor 3.

The previous detailed description is provided to enable any person skilled in the art to make or use the present invention. Various modifications to those embodiments will be readily apparent to those skilled in the art, and the generic principles defined herein may be applied to other embodiments without departing from the spirit or scope of the invention described herein. Thus, the present invention is not intended to be limited to the embodiments shown herein, but is to be accorded the full scope consistent with the claims, wherein reference to an element in the singular, such as by use of the article “a” or “an” is not intended to mean “one and only one” unless specifically so stated, but rather “one or more”. All structural and functional equivalents to the elements of the various embodiments described throughout the disclosure that are known or later come to be known to those of ordinary skill in the art are intended to be encompassed by the elements of the claims. Moreover, nothing disclosed herein is intended to be dedicated to the public regardless of whether such disclosure is explicitly recited in the claims.

1. Use of a compound of formula I as a shale hydration inhibition agent: 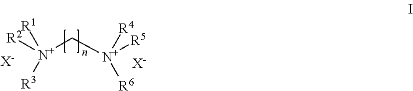
wherein R¹, R², R³, R⁴, R⁵ and R⁶ are each independently selected from hydrogen, (C₁₋₆)alkyl, (C₂₋₆)alkenyl, (C₂₋₆)alkynyl, (C₃₋₇)cycloalkyl, hydroxy(C₁₋₆)alkyl, (C₁₋₆)alkoxy(C₁₋₆)alkyl, aryl(C₁₋₆)alkyl, and (C₁₋₆)alkylamido(C₁₋₆)alkyl;
n is an integer from 1 to 6; and
X⁻ is a counterion;
provided that when n is 6, at least one of R¹, R², R³, R⁴, R⁵ and R⁶ is not hydrogen.

2. The use according to claim 1 wherein R¹, R², R³, R⁴, R⁵ and R⁶ are each independently selected from hydrogen, (C₁₋₄)alkyl, (C₂₋₄)alkenyl, (C₂₋₄)alkynyl, (C₃₋₆)cycloalkyl, hydroxy(C₁₋₄)alkyl, (C₁₋₄)alkoxy(C₁₋₄)alkyl, aryl(C₁₋₄)alkyl, and (C₁₋₄)alkylamido(C₁₋₄)alkyl.

3. The use according to claim 1 or 2 wherein n is an integer from 1 to 4.

4. The use according to claim 1 or 2 wherein n is an integer selected from 1, 2, 4 and 5.

5. The use according to any one of claims 1 to 4 wherein X⁻ is a counterion selected from bromide, chloride, iodide, hydroxide, a carboxylate, and a sulfonate.

6. The use according to any one of claims 1 to 5 wherein the group —N⁺(R¹)(R²)(R³) is the same as the group —N⁺(R⁴)(R⁵)(R⁶).

7. The use according to any one of claims 1 to 5 wherein the group —N⁺(R¹)(R²)(R³) is different from the group —N⁺(R⁴)(R⁵)(R⁶).

8. The use according to claim 1 wherein the compound of formula I has the formula:

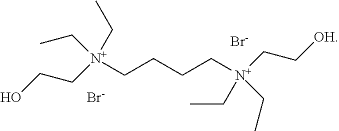

9. The use according to claim 1 wherein the compound of formula I has the formula:

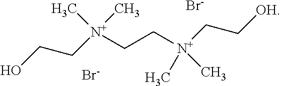

10. The use according to claim 1 wherein the compound of formula I has the formula:

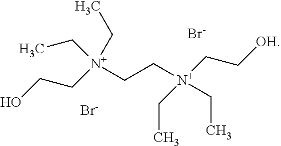

11. The use according to claim 1 wherein the compound of formula I has the formula:

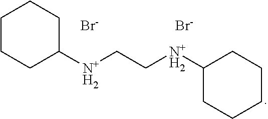

12. The use according to claim 1 wherein the compound of formula I has the formula:

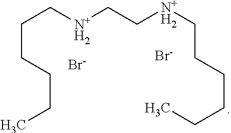

13. The use according to claim 1 wherein the compound of formula I has the formula:

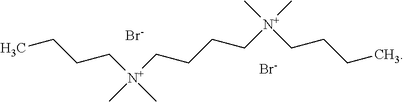

14. The use according to claim 1 wherein the compound of formula I has the formula:

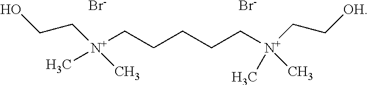

15. A water-based drilling fluid comprising a compound of formula I according to any one of claims 1 to 14.

16. The water-based drilling fluid according to claim 15 wherein the drilling fluid further comprises a weight material.

17. The water based drilling fluid according to claim 15 or 16 wherein the drilling fluid further comprises at least one fluid loss control agent, at least one bridging agent, at least one lubricant, at least one anti-bit balling agent, at least one corrosion inhibition agent, at least one surfactant or at least one suspending agent.

18. Use of a compound of formula I according to any one of claims 1 to 14 to inhibit the hydration or swelling of shale when drilling through a formation containing shale.

19. A method of inhibiting the hydration and swelling of shale when drilling through a formation containing shale, the method comprising using a water based drilling fluid according to any one of claims 15 to 17.
